(19) United States
(12) Patent Application Publication
(10) Pub. No.: US 2007/0243540 A1
(43) Pub. Date: Oct. 18, 2007

(54) Kits and Methods for Identification, Assessment, Prevention and Therapy of Breast Cancer

(76) Inventors: Jo C. Tsai, West Roxbury, MA (US); William S. Hancock, Brookline, MA (US); Barry L. Karger, Newton, MA (US)

Correspondence Address: WEINGARTEN, SCHURGIN, GAGNEBIN & LEBOVICI LLP, TEN POST OFFICE SQUARE, BOSTON, MA, 02109, US

(21) Appl. No.: 11/597,159

(22) PCT Filed: May 23, 2005
(86) PCT No.: PCT/US05/18033

§ 371 Date: Nov. 21, 2006

Related U.S. Application Data
(60) Provisional application No. 60/573,282, filed on May 21, 2004.

(51) Int. Cl. G01N 33/53; C12Q 1/68

(52) U.S. Cl. 435/6; 435/7.23

(57) ABSTRACT

Disclosed herein are methods for detecting and identifying potential breast cancer biomarkers in an individual patient. Also disclosed are newly discovered breast cancer markers set forth in Tables I, II and III, associated with the cancerous state of breast cells. It has been discovered that a higher than normal level of expression of any of these markers or combination of these markers correlates with breast cancer in a patient. Methods are provided for detecting the presence of breast cancer in a sample, the absence of breast cancer in a sample, the stage of breast cancer, assessing whether a breast cancer has metastasized, predicting the likely clinical outcome of a breast cancer patient, and with other characteristics of breast cancer that are relevant to prevention, diagnosis, characterization, and therapy of breast cancer in a patient.

### CROSS REFERENCE TO RELATED APPLICATIONS

This application claims the priority of U.S. Provisional Application No. 60/573,282 filed May 21, 2004 entitled, PROTEOMIC METHODS FOR IDENTIFYING POTENTIAL MARKERS FOR BREAST CANCER, the whole of which is hereby incorporated by reference herein.

### STATEMENT REGARDING FEDERALLY SPONSORED RESEARCH OR DEVELOPMENT

N/A

### BACKGROUND OF THE INVENTION

Breast cancer is the most common fatal malignancy in women, and about 15% of all women will be diagnosed with breast cancer during their lifetime. Despite recent progress in early detection as well as improved treatment, the mortality rate remains unchanged. Early detection and diagnosis of cellular transformation and tumor formation in breast tissue is the key to surviving breast cancer.

The majority of breast cancers originate in the epithelium lining the ductal system of the breast. The cells of the lobular and ductal regions of the breast secrete proteins directly into the ducts, and secreted fluid in the ducts can be collected with a suction device. This fluid is known as nipple aspirate fluid (NAF). Analysis of pooled samples for the biochemical and cellular content of NAF has recently gained attention as a method for studying the local microenvironment associated with the development and progression of breast cancer.

Alternatively, the secreted proteins of the lobular and ductal regions can be collected as a lavage of individual ducts termed as ductal lavage (DL). Ductal lavage is a minimally invasive procedure performed on women who are considered to be at high risk for breast carcinoma, e.g., who have a family history of breast cancer. To this end, breast ductal epithelial cells have been collected for cytologic analysis of atypia to provide further risk stratification. A sample collected by ductal lavage contains a larger number of cells than a NAF sample and, therefore, is more useful for cytological analysis. (See, U.S. Pat. No. 6,642,009, which is hereby incorporated by reference herein.)

Currently, the principal manner of identifying breast cancer is through detection of the presence of dense tumorous tissue. This may be accomplished to varying degrees of effectiveness by direct examination of the outside of the breast, or through mammography or other X-ray imaging methods. The latter approach is not without considerable cost, however. It would, therefore, be beneficial to provide specific methods and reagents for the diagnosis, staging, prognosis, monitoring and treatment of diseases associated with breast cancer or to indicate a predisposition to such for preventative measures.

### BRIEF SUMMARY OF THE INVENTION

Provided herein are panels of proteins identified in ductal lavage samples from individual women at high risk of developing breast cancer. The proteins in these panels, either individually or as relative ratios, are potential biomarkers for the identification of a precancerous condition or of breast cancer itself. Also disclosed are proteomic methods to detect and to identify potential biomarkers using, e.g., ductal lavage from individual ducts, or pooled ducts, of each breast of an individual subject. The analysis of protein profiles in individual ducts is the most accurate way of determining the extent of an existing cancer. Alternatively, it may be preferred to pool all lavages from a single breast. In this way, the variability of individual ducts is averaged and, as well, an overall view of the potential pathology of the breast is obtained. Ductal lavage provides a minimally invasive procedure to study the local microenvironment associated with development and progression of breast tumors.

Proteomic analysis of ductal lavage samples using a sensitive analytical method such as liquid chromatography/mass spectrometry (LC/MS) gives a snapshot of the protein components in the ductal lavage. Using the methods of the invention, potential biomarker proteins for breast cancer, or a predisposition to breast cancer, have been characterized in individual subjects for the first time. Use of proteins identified by the method of the invention as potential biomarkers is within the invention. Thus, the invention provides a sensitive method for early detection, diagnosis and monitoring of breast cancer not only by comparing the protein profiles of the ductal lavage from each breast of an individual to a control sample, but also by detecting differences among individual ducts of the breast. For example, the methods of the present invention can be of use in identifying patients having an enhanced risk of developing breast cancer (e.g., patients having a familial history of breast cancer, patients identified as having a mutant oncogene). The methods are also useful diagnostics for assessing whether a patient has an aggressive breast cancer or is likely to develop an aggressive breast tumor. In addition, the methods of the invention are useful for distinguishing benign pathologies from malignant pathologies, e.g., through the differences in protein profiles.

The invention additionally provides a test method of assessing the breast carcinogenic potential of a compound. This method comprises the steps of: maintaining separate aliquots of breast cells in the presence and absence of a compound; and comparing expression of a marker of the invention in each of the aliquots. A significantly higher level of expression of the marker in the aliquot maintained in the presence of the compound, relative to that of the aliquot maintained in the absence of the compound, is an indication that the compound possesses breast carcinogenic potential.

In addition, the invention further provides a method of assessing the potential of a compound as an inhibitor of breast cancer in a patient. This method comprises the steps of obtaining a sample comprising cancer cells from a patient; separately maintaining at least one sample comprising cancer cells from a patient in the presence of a test composition; comparing expression of a marker of the invention in each of the aliquots; and identifying a composition as an inhibitor of breast cancer where the composition significantly lowers the level of expression of a marker of the invention in the aliquot containing the composition relative to the levels of expression of the marker in the presence of the other compositions. Compositions so identified can be administered to a patient having breast cancer for treating or for inhibiting the further development of the breast cancer.

Still further, the methods of the present invention are also useful for predicting the clinical outcome for a patient with breast cancer, or for a patient who has undergone therapy to eradicate breast cancer. Additionally, the methods of the present invention are also useful in assessing the efficacy of treatment of a breast cancer patient (e.g., the efficacy of chemotherapy).

It will be appreciated that in these methods the “therapy” may be any therapy for treating breast cancer including, but not limited to, chemotherapy, radiation therapy, surgical removal of tumor tissue, gene therapy and biologic therapy, such as the administering of antibodies and chemokines. Thus, the methods of the invention may be used to evaluate a patient before, during and after therapy, for example, to evaluate the reduction in tumor burden.

In another aspect, the invention relates to various diagnostic and test kits. In one embodiment, the invention provides a kit for assessing whether a patient is afflicted with a breast tumor. In another aspect, the kit may be used for assessing whether a patient is at risk of developing a breast tumor. The kit comprises a reagent for assessing expression of at least one marker of the invention. The kit comprises a reagent for assessing expression of at least one marker of the invention. In another embodiment, the invention provides a kit for assessing the suitability of a chemical or biologic agent for inhibiting breast cancer in a patient. Such a kit comprises reagents for assessing expression of at least one marker of the invention and may also comprise one or more of such agents. In a further embodiment, the invention provides kits for assessing the presence of breast cancer cells or treating breast cancers. Such kits may comprise an antibody, an antibody derivative, or an antibody fragment that binds specifically with a marker protein, or a fragment of the protein. Such kits may also comprise a plurality of antibodies, antibody derivatives, or antibody fragments wherein the plurality of such antibody agents binds specifically with a marker protein, or a fragment of the protein. It will be appreciated that the methods and kits of the present invention may also include known breast cancer markers.

Other features and advantages of the invention will be apparent from the following description of the preferred embodiments thereof and from the claims.

### DETAILED DESCRIPTION OF THE INVENTION

The invention relates to methods for detecting and identifying potential breast cancer biomarkers in an individual patient. The invention also relates to newly discovered breast cancer markers set forth in Tables I, II and III, associated with the cancerous state of breast cells. It has been discovered that a higher than normal level of expression of any of these markers or combination of these markers correlates with breast cancer in a patient. Methods are provided for detecting the presence of breast cancer in a sample, the absence of breast cancer in a sample, the stage of breast cancer, assessing whether a breast cancer has metastasized, predicting the likely clinical outcome of a breast cancer patient, and with other characteristics of breast cancer that are relevant to prevention, diagnosis, characterization, and therapy of breast cancer in a patient.

As used herein, each of the following terms has the meaning associated with it in this section.

A “marker” is a protein, or associated gene, whose altered level of expression (or abundance) in a tissue or cell from its expression level in normal or healthy tissue or cell is associated with a disease state, such as cancer.

The term “probe” refers to any molecule that is capable of selectively binding to a specifically intended target molecule, for example, a nucleotide transcript or marker protein. Probes can be either synthesized by one skilled in the art or derived from appropriate biological preparations. For purposes of detection of the target molecule, probes may be specifically designed to be labeled, as described herein. Examples of molecules that can be utilized as probes include, but are not limited to, RNA, DNA, proteins, antibodies, and organic molecules.

“Breast cancer” as used herein includes carcinomas, (e.g., carcinoma in situ, invasive carcinoma, metastatic carcinoma) and pre-malignant conditions.

A “breast-associated” body fluid is a fluid that, when in the body of a patient, contacts or passes through breast cells or into which cells, nucleic acids or proteins shed from breast cells are capable of passing. Exemplary breast-associated body fluids include blood fluids, lymph, cystic fluid, nipple aspirates and ductal lavage.

A “sample” or “patient sample” comprises cells obtained from the patient, e.g., a lump biopsy, body fluids including blood fluids, lymph and cystic fluids, as well as nipple aspirates and ductal lavage. In a further embodiment, the patient sample is in vivo.

The “normal” level of expression of a marker is the level of expression of the marker in breast cells of a human subject or patient not afflicted with breast cancer.

An “over-expression” or “significantly higher level of expression” of a marker refers to an abundance or expression level in a test sample that is greater than the standard error of the assay employed to assess expression, and is preferably at least twice, and more preferably three, four, five or ten times the expression level of the marker in a control sample (e.g., sample from a healthy subjects not having the marker associated disease) and preferably, the average expression level of the marker in several control samples.

A “significantly lower level of expression” of a marker refers to an expression level in a test sample that is at least twice, and more preferably three, four, five or ten times lower than the expression level of the marker in a control sample (e.g., sample from a healthy subjects not having the marker associated disease) and preferably, the average expression level of the marker in several control samples.

A cancer is “inhibited” if at least one symptom of the cancer is alleviated, terminated, slowed, or prevented. As used herein, breast cancer is also “inhibited” if recurrence or metastasis of the cancer is reduced, slowed, delayed, or prevented.

A “kit” is any manufacture (e.g., a package or container) comprising at least one reagent, e.g., a probe, for specifically detecting the abundance or expression of a marker of the invention. The kit may be promoted, distributed, or sold as a unit for performing the methods of the present invention.

“Proteins of the invention” encompass marker proteins and their fragments; variant marker proteins and their fragments; peptides and polypeptides comprising an at least 15 amino acid segment of a marker or variant marker protein; and fusion proteins comprising a marker or variant marker protein, or an at least 15 amino acid segment of a marker or variant marker protein.

Unless otherwise specified herein, the terms “antibody” and “antibodies” broadly encompass naturally-occurring forms of antibodies (e.g., IgG, IgA, IgM, IgE) and recombinant antibodies such as single-chain antibodies, chimeric and humanized antibodies and multi-specific antibodies, as well as fragments and derivatives of all of the foregoing, which fragments and derivatives have at least an antigenic binding site. Antibody derivatives may comprise a protein or chemical moiety conjugated to an antibody.

The present invention is based, in part, on newly identified markers which are over-expressed in breast cancer cells as compared to their expression in normal (i.e., non-cancerous) breast cells. The enhanced expression of one or more of these markers in breast cells is herein correlated with the cancerous state of the tissue. The invention provides compositions, kits, and methods for assessing the cancerous state of breast cells (e.g., cells obtained from a human, cultured human cells, archived or preserved human cells and in vivo cells) as well as treating patients afflicted with breast cancer.

The compositions, kits, and methods of the invention have the following uses, among others:

assessing the status of breast cancer in a human patient; assessing the stage of breast cancer in a human patient; assessing the grade of breast cancer in a patient; assessing the benign or malignant nature of breast cancer in a patient; assessing the metastatic potential of breast cancer in a patient; determining if breast cancer has metastasized to lymph nodes; predicting the clinical outcome of a breast cancer patient; assessing whether a patient is afflicted with breast cancer; assessing the histological type of neoplasm associated with breast cancer in a patient; making antibodies, antibody fragments or antibody derivatives that are useful for treating breast cancer and/or assessing whether a patient is afflicted with breast cancer; assessing the presence of breast cancer cells; assessing the efficacy of one or more test compounds for inhibiting breast cancer in a patient; assessing the efficacy of a therapy for inhibiting breast cancer in a patient; monitoring the progression of breast cancer in a patient; selecting a composition or therapy for inhibiting breast cancer in a patient; treating a patient afflicted with breast cancer; inhibiting breast cancer in a patient; assessing the breast carcinogenic potential of a test compound; and preventing the onset of breast cancer in a patient at risk for developing breast cancer.

The invention thus includes a method of assessing breast cancer cells in a patient afflicted with breast cancer. This method comprises comparing the level of expression of a marker of the invention (listed in Tables I, II and III) in a patient sample and the normal level of expression of the marker in a control, e.g., a non-breast cancer sample or a non-cancer, normal sample. A significantly higher level of expression of the marker in the patient sample as compared to the normal level of expression is an indication that the patient is afflicted with a breast tumor.

As described herein, breast cancer in patients is associated with the qualitative appearance of or an increased level of expression of one or more markers of the invention. While, as discussed above, some of these changes in expression level result from occurrence of the breast cancer, others of these changes induce, maintain, and promote the cancerous state of breast cancer cells. Thus, breast cancer characterized by an increase in the level of expression of one or more markers of the invention can be inhibited by reducing and/or interfering with the expression of the markers and/or function of those markers. Expression of a marker of the invention can be inhibited in a number of ways generally known in the art.

Any marker or combination of markers of the invention, as well as any known markers in combination with the markers of the invention, may be used in the compositions, kits, and methods of the present invention. In general, it is preferable to use markers for which, in quantitative terms, the difference between the level of expression of the marker in breast cancer cells and the level of expression of the same marker in normal breast cells is as great as possible. Although this difference can be as small as the limit of detection of the method for assessing expression of the marker or as large as the qualitative difference between the presence and the absence of the marker, it is preferred that the difference be at least greater than the standard error of the assessment method, and preferably a difference of at least 2-, 3-, 4-, 5-, 6-, 7-, 8-, 9-, 10-, 15-, 20-, 25-, 100-, 500-, 1000-fold or greater than the level of expression of the same marker in normal breast tissue.

It is recognized that certain marker proteins are secreted from breast cells (i.e., one or both of normal and cancerous cells) to the extracellular space surrounding the cells. These markers are preferably used in certain embodiments of the compositions, kits, and methods of the invention, owing to the fact that the such marker proteins can be detected in a breast-associated body fluid sample, which may be more easily collected from a human patient than a tissue biopsy sample. In addition, preferred in vivo techniques for detection of a marker protein include introducing into a subject a labeled antibody directed against the protein. For example, the antibody can be labeled with a radioactive marker whose presence and location in a subject can be detected by standard imaging techniques.

It will be appreciated that patient samples containing breast cells may be used in the methods of the present invention. In these embodiments, the level of expression of the marker can be determined by assessing the amount (e.g., absolute amount or concentration) of the marker in a breast cell sample, e.g., breast biopsies obtained from a patient. The cell sample can, of course, be subjected to a variety of well-known post-collection preparative and storage techniques (e.g., nucleic acid and/or protein extraction, fixation, storage, freezing, ultrafiltration, concentration, evaporation, centrifugation, etc.) prior to assessing the amount of the marker in the sample. Likewise, breast biopsies may also be subjected to post-collection preparative and storage techniques, e.g., fixation.

Expression or abundance of a marker of the invention may be assessed by any of a wide variety of well known methods for detecting expression of a protein or its encoding nucleic acid. Non-limiting examples of such methods include immunological methods for detection of secreted, cell-surface, cytoplasmic, or nuclear proteins, protein purification methods, protein function or activity assays, nucleic acid hybridization methods, nucleic acid reverse transcription methods, and nucleic acid amplification methods. Preferably, the assessments include the highly sensitive proteomic techniques described herein.

When a plurality of markers of the invention are used in the compositions, kits, and methods of the invention, the level of expression of each marker in a patient sample can be compared with the normal level of expression of each of the plurality of markers in non-cancerous samples of the same type, either in a single reaction mixture (i.e., using reagents, such as different fluorescent probes, for each marker) or in individual reaction mixtures corresponding to one or more of the markers. In one embodiment, a significantly increased level of expression of more than one of the plurality of markers in the sample, relative to the corresponding normal levels, is an indication that the patient is afflicted with breast cancer. When a plurality of markers is used, it is preferred that 2, 3, 4, 5, 8, 10, 12, or 15, or more individual markers be used. Still further markers can be used to include a marker set wherein at least 20, 25, 30, 40, 50, or more individual markers are used.

In order to maximize the sensitivity of the compositions, kits, and methods of the invention, it is preferable that the marker of the invention used therein be a marker that has a restricted tissue distribution, e.g., normally not expressed in a non-breast tissue.

Only a small number of markers are known to be associated with breast cancers (e.g., BRCA1 and BRCA2). These markers are not, of course, included among the markers of the invention, although they may be used together with one or more markers of, the invention in a panel of markers, for example. It is well known that certain types of genes, such as oncogenes, tumor suppressor genes, growth factor-like genes, protease-like genes, and protein kinase-like genes are often involved with development of cancers of various types. Thus, among the markers of the invention, use of those which correspond to proteins which resemble known proteins encoded by known oncogenes and tumor suppressor genes, and those which correspond to proteins which resemble growth factors, proteases, and protein kinases are preferred.

It is recognized that the compositions, kits, and methods of the invention will be of particular utility to patients having an enhanced risk of developing breast cancer and their medical advisors. Patients recognized as having an enhanced risk of developing breast cancer include, for example, patients having a familial history of breast cancer, patients identified as having a mutant oncogene (i.e., at least one allele), and patients of advancing age (i.e., women older than about 50 or 60 years).

The level of expression of a marker in normal (i.e., non-cancerous) breast tissue can be assessed in a variety of ways. In one embodiment, this normal level of expression is determined by assessing the level of expression of the marker in a portion of breast cells which appears to be non-cancerous and by comparing this normal level of expression with the level of expression in a portion of the breast cells which is suspected of being cancerous. Alternately, and particularly as further information becomes available as a result of routine performance of the methods described herein, population-average values for normal expression of the markers of the invention may be used. In other embodiments, the “normal” level of expression of a marker may be determined by assessing expression of the marker in a patient sample obtained from a non-cancer-afflicted patient, from a patient sample obtained from a patient before the suspected onset of breast cancer in the patient, from archived patient samples, and the like.

The invention also includes a method of assessing the efficacy of a test compound for inhibiting breast cancer cells. As described above, differences in the level of expression of the markers of the invention correlate with the cancerous state of breast cells. Although it is recognized that changes in the levels of expression of certain of the markers of the invention likely result from the cancerous state of breast cells, it is likewise recognized that changes in the levels of expression of other of the markers of the invention induce, maintain, and promote the cancerous state of those cells. Thus, compounds which inhibit a breast cancer in a patient will cause the level of expression of one or more of the markers of the invention to change to a level nearer the normal level of expression for that marker (i.e., the level of expression for the marker in non-cancerous breast cells).

This method thus comprises comparing expression of a marker in a first breast cell sample and maintained in the presence of the test compound and expression of the marker in a second breast cell sample and maintained in the absence of the test compound. A significantly reduced expression of a marker of the invention in the presence of the test compound is an indication that the test compound inhibits breast cancer. The breast cell samples may, for example, be aliquots of a single sample of normal breast cells obtained from a patient, pooled samples of normal breast cells obtained from a patient, cells of a normal breast cell line, aliquots of a single sample of breast cancer cells obtained from a patient, pooled samples of breast cancer cells obtained from a patient, cells of a breast cancer cell line, or the like. In one embodiment, the samples are breast cancer cells obtained from a patient and one or more of a plurality of compounds known to be effective for inhibiting various breast cancers are tested in order to identify the compound which is likely to best inhibit the breast cancer in the patient.

This method may likewise be used to assess the efficacy of a therapy for inhibiting breast cancer in a patient. In this method, the level of expression of one or more markers of the invention in a pair of samples (one subjected to the therapy, the other not subjected to the therapy) is assessed. As with the method of assessing the efficacy of test compounds, if the therapy induces a significantly lower level of expression of a marker of the invention, then the therapy is efficacious for inhibiting breast cancer. As above if samples from a selected patient are used in this method, then alternative therapies can be assessed in vitro in order to select a therapy most likely to be efficacious for inhibiting breast cancer in the patient.

As described above, the cancerous state of human breast cells is correlated with changes in the levels of expression of the markers of the invention. The invention includes a method for assessing the human breast cell carcinogenic potential of a test compound. This method comprises maintaining separate aliquots of human breast cells in the presence and absence of the test compound. Expression of a marker of the invention in each of the aliquots is compared. A significantly higher level of expression of a marker of the invention in the aliquot maintained in the presence of the test compound (relative to the aliquot maintained in the absence of the test compound) is an indication that the test compound possesses human breast cell carcinogenic potential. The relative carcinogenic potentials of various test compounds can be assessed by comparing the degree of enhancement or inhibition of the level of expression of the relevant markers, by comparing the number of markers for which the level of expression is enhanced or inhibited, or by comparing both.

The present invention pertains to the field of predictive medicine in which diagnostic assays, prognostic assays, pharmacogenomics, and monitoring clinical trails are used for prognostic (predictive) purposes to thereby treat an individual prophylactically. Accordingly, one aspect of the present invention relates to diagnostic assays for determining the level of expression of one or more marker proteins or nucleic acids, in order to determine whether an individual is at risk of developing breast cancer. Such assays can be used for prognostic or predictive purposes to thereby prophylactically treat an individual prior to the onset of the cancer. Yet another aspect of the invention pertains to monitoring the influence of agents (e.g., drugs or other compounds administered either to inhibit breast cancer or to treat or prevent any other disorder, in order to understand any breast carcinogenic effects that such treatment may have) on the expression or activity of a marker of the invention in clinical trials.

An exemplary method for detecting the presence or absence of a marker protein or nucleic acid in a biological sample involves obtaining a biological sample (e.g., a breast associated body fluid) from a test subject and contacting the biological sample with a compound or an agent capable of detecting the polypeptide or nucleic acid (e.g., mRNA, genomic DNA, or cDNA). The detection methods of the invention can thus be used to detect protein, mRNA, cDNA, or genomic DNA, for example, in a biological sample in vitro as well as in vivo.

A general principle of such diagnostic and prognostic assays involves preparing a sample or reaction mixture that may contain a marker, and a probe, under appropriate conditions and for a time sufficient to allow the marker and probe to interact and bind, thus forming a complex that can be removed and/or detected in the reaction mixture. These assays can be conducted in a variety of ways known to those of ordinary skill in the art, and particularly as described herein.

The invention also encompasses kits for detecting the presence of a marker protein or nucleic acid in a biological sample (e g., a breast-associated body fluid such as a nipple aspirate). Such kits can be used to determine if a subject is suffering from or is at increased risk of developing breast cancer. For example, the kit can comprise a labeled compound or agent capable of detecting a marker protein or nucleic acid in a biological sample and means for determining the amount of the protein or mRNA in the sample (e.g., an antibody which binds the protein or a fragment thereof, or an oligonucleotide probe which binds to DNA or mRNA encoding the protein). Kits can also include instructions for interpreting the results obtained using the kit.

Thus, the level of expression of a marker of the invention in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a modulator of expression of a marker of the invention.

Monitoring the influence of agents (e.g., drug compounds) on the level of expression of a marker of the invention can be applied not only in basic drug screening, but also in clinical trials. For example, the effectiveness of an agent to affect marker expression can be monitored in clinical trials of subjects receiving treatment for breast cancer. In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate) comprising the steps of (i) obtaining a pre-administration sample from a subject prior to administration of the agent; (ii) detecting the level of expression of one or more selected markers of the invention in the pre-administration sample; (iii) obtaining one or more post-administration samples from the subject; (iv) detecting the level of expression of the marker(s) in the post-administration samples; (v) comparing the level of expression of the marker(s) in the pre-administration sample with the level of expression of the marker(s) in the post-administration sample or samples; and (vi) altering the administration of the agent to the subject accordingly. For example, increased expression or abundance of marker(s) during the course of treatment may indicate ineffective dosage and the desirability of increasing the dosage. Conversely, decreased expression of the marker(s) may indicate efficacious treatment and no need to change dosage.

The invention also includes an array comprising a marker of the present invention. The array can be used to assay abundance of, e.g., one or more proteins in the array. In one embodiment, the array can be used to assay protein abundance in ductal lavage from an individual duct to ascertain the specificity of proteins in the array. In this manner, a large number of proteins can be simultaneously assayed for expression or abundance level. This allows a profile to be developed showing a battery of proteins specifically expressed in one or more ducts.

In addition to such qualitative determination, the invention allows the quantitation of protein expression. Thus, not only duct specificity, but also the level of abundance of a battery of proteins in the duct is ascertainable. Thus, proteins can be grouped on the basis of their expression site per se and level of expression at that site.

In another embodiment, the array can be used to monitor the time course of expression of one or more proteins in the array. This can occur in various biological contexts, as disclosed herein, for example, development of breast cancer, progression of breast cancer, and processes such a cellular transformation associated with breast cancer.

Disorders of the breast include, but are not limited to, disorders of development; inflammations, including but not limited to, acute mastitis, periductal mastitis, periductal mastitis (recurrent subareolar abscess, squamous metaplasia of lactiferous ducts)., mammary duct ectasia, fat necrosis, granulomatous mastitis, and pathologies associated with silicone breast implants; fibrocystic changes; proliferative breast disease including, but not limited to, epithelial hyperplasia, sclerosing adenosis, and small duct papillomas; tumors including, but not limited to, stromal tumors such as fibroadenoma, phyllodes tumor, and sarcomas, and epithelial tumors such as large duct papilloma; carcinoma of the breast including in situ (noninvasive) carcinoma that includes ductal carcinoma in situ (including Paget's disease) and lobular carcinoma in situ, and invasive (infiltrating) carcinoma including, but not limited to, invasive ductal carcinoma, no special type, invasive lobular carcinoma, medullary carcinoma, colloid (mucinous) carcinoma, tubular carcinoma, and invasive papillary carcinoma, and miscellaneous malignant neoplasms. Disorders in the male breast include, but are not limited to, gynecomastia and carcinoma.

Premalignant and malignant lesions are usually confined to the breast ductal system and the terminal ductal lobular unit. The terminal ductal lobular unit or TDLU is the network of ducts and ductal tributaries located at and towards the base of the breast. This network flows into the milk ducts of the breast that extend from the TDLU towards the nipple. Ultimately, the milk ducts each end at a ductal orifice located on the nipple surface. Women have an average of 6 to 12 ductal orifices on each nipple. For description and definition of terminal ductal lobular unit, see Wellings S R, Pathol Res Pract 166(4): 515-35 (1980), Stirling and Chandler, Virchows Arch A Pathol Anat Histol 372 (3): 205-26 (1976), and Fraser et al., Am J Surg Pathol 22(12): 1521-7 (1998).

Breast cancer usually begins in the cells lining a breast duct and in the terminal ductal lobular unit, with the first stage thought to be excessive proliferation of individual cell(s) leading to “ductal hyperplasia.” Some of the hyperplastic cells may then become atypical, with a significant risk of the atypical hyperplastic cells becoming neoplastic or cancerous. Initially, the cancerous cells remain in the breast ducts, and the condition is referred to as ductal carcinoma in situ (DCIS). After a time, however, the cancerous cells are able to invade outside of the ductal environment, presenting the risk of metastases which can be fatal to the patient. Breast cancer proceeds through discrete premalignant and malignant cellular stages: normal ductal epithelium, atypical ductal hyperplasia, ductal carcinoma in situ, and finally invasive ductal carcinoma. The first three stages are confined within the ductal system, including the terminal ductal lobular unit, and therefore if diagnosed and treated, offer the greatest probability of cure. All of these stages can be characterized by unique cellular markers.

While breast cancer through the DCIS phase is in theory quite treatable, effective treatment requires both early diagnosis and an effective treatment modality. At present, mammography is the state-of-the-art diagnostic tool for detecting breast cancer. Often, however, mammography is only able to detect tumors that have reached a size in the range from 0.1 cm to 1 cm. Such a tumor mass may be reached as long as from 8 to 10 years following initiation of the disease process. Detection of breast cancer at such a late stage is often too late to permit effective treatment.

Previously to this invention, it was believed by those skilled in the art that the protein contents of samples obtainable by ductal lavage were too small or too diluted for successful biochemical analysis and protein identification in individual patients, a necessary condition in the search for diagnostic biomarkers. However, using the method of the invention, it has been determined that the biochemical microenvironment within the breast can be readily accessed and evaluated by ductal lavage. Thus, the secreted/shedded proteins in ductal lavage are a good source of potential diagnostic biomarkers. Identification of ductal proteins will lead to better understanding of breast physiology and pathophysiology and, thus, to the discovery of novel biomarkers for breast diseases. Protein expression profiling comparing samples from healthy individuals and cancer patients will be useful for identifying clinically relevant tumor markers for risk stratification, diagnosis, treatment monitoring, and detection of cancer recurrence.

The most powerful information obtained from this type of analysis includes the identification of protein expression profiles that become apparent early in breast carcinogenesis, before a tumor is detectable by physical examination or radiologic imaging. Identification of proteins that are secreted in response to carcinogenesis can also be extremely valuable in elucidation of the biology of breast cancer. Potential protein biomarkers discovered in ductal fluids can be used to identify women who are at high risk for the development of breast cancer and to monitor disease progression and/or the efficacy of a therapeutic treatment over time.

The ability to examine the expression of a majority of breast ductal proteins simultaneously in individual woman represents a significant technical advance and will permit the independent analysis of ductal carcinoma in situ as distinguished from lobular carcinoma. The method of the invention makes it possible to avoid the limitations of antibody-based studies and provides an opportunity to study post-translational modifications of these proteins. Although proteomic studies carried out on NAF samples yielded a handful of protein identifications, the utility of such studies was limited because they were based on pooled NAF samples from a number of subjects. Two-dimensional gel electrophoresis (2D-gel) was carried out to compare individual NAF samples for differential expression between normal and breast cancer patients, but while differences in spot intensities have been observed, this approach has not resulted in protein identifications because of the difficulty of extracting low level proteins from gel spots. Expression of diagnostic marker proteins in NAF has been previously studied by ELISA and other immunological methods. The scope of such studies, however, is limited to previously identified proteins for which specific antibodies exist.

The following examples are presented to illustrate the advantages of the present invention and to assist one of ordinary skill in making and using the same. These examples are not intended in any way otherwise to limit the scope of the disclosure.

Ductal lavage is a minimally invasive procedure carried out to collect breast ductal epithelial cells for cytological analysis. The procedure involves inserting a microcatheter approximately 1.5 cm into a nipple orifice after administration of topical anesthesia; lavaging the cannulated ductal system with normal saline; and analyzing the collected lavage effluent for the presence of normal, atypical, or malignant breast ductal cells. Ductal lavage was collected from high risk patients and centrifuged to separate the cellular and fluid fractions. The supernatants (fluid fractions) were used for proteomic analysis. For each DL sample, protein concentration was determined by a micro BCA assay (Pierce) and ranged between 2 μg/ml and 300 μg/ml. Depending on the sampling strategy (see above) in most cases, individual ducts of the same breast were combined for the analysis. For proteomic studies using the more sensitive LC/MS method, ductal lavage samples were denatured, reduced and alkylated prior to tryptic digestion. The tryptic peptide mixture of DL (between 2 μg and 6 μg) was separated on a narrow i.d. (e.g., 75 μm) capillary C18 reversed-phase (RP)-HPLC column coupled online either to an ion-trap (3D-trap) mass spectrometer (MS) (LCQ Deca XP, ThermoFinnegan) or to a newer linear ion-trap (2D-trap) MS, with an electrospray ionization interface. The SEQUEST algorithm was used to match the raw mass spectra against the theoretically calculated spectra generated from the human protein database (e.g., SwissProt). A protein was considered as identified by using the conservative criteria developed by Yates (Xcorr and delta Cn) (e.g., as disclosed in An Evaluation of Shotgun Sequencing for Proteomic Analysis of Human Plasma using HPLC Coupled With Either Ion Trap or Fourier Transform Mass Spectrometry, Wu, S.-L, Choudhary, G., Ramstrom, M., Bergquist, J. and Hancock, W. S., (2003) J. Proteome Res., 2, 383-393) and by manual inspection of the MS/MS spectra. Proteins identified with at least two peptide MS/MS scans are shown in Table I. The proteins identified from 2D-trap MS were further filtered through the Protein Prophet (a probability based algorithm developed by the Institute of System Biology, Seattle, Wash. as public shareware, and those with higher than 95% probability (indicating high confidence) are listed in Table II.

### EXAMPLE I

### Protein Profiling of Ductal Lavage Samples

Ductal lavage samples from individual patients were examined for protein expression (or abundance) profiling. Unlike nipple aspirate fluid (NAF), which represents a pooled fluid fraction, ductal lavage can be reflective of the microenvironment of individual ducts in the breast. In general, however, DL samples within an individual breast were pooled for easier sample handling. It is important to understand what types of proteins have been secreted to the breast fluids, and characterization of the proteins present in the ductal lavage is a prerequisite for identifying potential breast cancer markers. Use of both the 3D-trap and 2D-trap MS instruments provided a sensitive method for detecting medium to low abundant proteins in DL. Moreover, by comparing the protein expression profiles of DL of individual patients and within individual ducts of the patient, enormous information can be amassed about the breast physiological and pathological status.

The protein concentration varied significantly among DL samples. Therefore, normalization of DL samples against total protein contents is required for differential quantitation studies. Due to the limited amount of protein available for each sample, only a few micrograms of proteins were analyzed by mass spectrometry. The “shotgun” proteomic approach by LC-MS has the advantage over 2D-gel electrophoresis because it is more sensitive and suitable for small quantities of protein samples. With the increased sensitivity of the LC/MS method and without the recovery issue of 2D gels, this approach is able to produce a protein profile for individual patients for individual risk assessment. By using this approach, a significant number of proteins were identified for each sample, and the proteins expressed in 10 individual subjects were combined to obtain a comprehensive protein profiling of DL. The proteins that were identified by at least two peptides using the 3D-trap MS and were present in most DL samples are listed in Table I.

The number of peptides identified for each protein was used as a rough estimate of its abundance. Some of the most abundant proteins identified by mass spectrometry were also correlated with the band pattern on a 1D SDS-PAGE gel. As expected, a large number of light chains and heavy chains of immunoglobulin (Ig) molecules, such as Ig alpha-1 and -2 chain C, Ig kappa chain C, Ig J chain, Ig lambda chain C, were found in most DL samples in high abundance. Excluding immunoglobulins, 19 proteins were common in many DL samples (Table I). There were a lot of similarities between the DL proteins and those reported for NAF, including serum albumin, lactotransferrin, apolipoprotein D (ApoD), polymeric Ig receptor, Zn-α-2-glycoprotein, complement C3 and C4, antitrypsin, antichymotrypsin, prolactin-inducible protein (PIP)/GCDFP-15 and clusterin. However, there were several unique proteins in DL, which did not appear to be abundant or present in NAF. Conversely, certain high abundant NAF proteins such as β-casein, a major milk protein, were absent in the DL samples. These data indicate that although both NAF and DL originate similarly from the breast ductal system, the protein composition appears to be different, and comparison should yield valuable information about the mammary gland microenvironment.

The abundant proteins in DL contained several classic plasma and secreted proteins, including serum albumin, immunoglobulins, apolipoprotein D, serotransferrin, complement C3 and C4, antitrypsin, antichymotrypsin, α-1 glycoproteins 1 and 2, and clusterin. However, the overall protein composition and relative abundance in DL were remarkably different from that of plasma/serum. Some of the secreted proteins in DL reflected the mammary gland specific origin, such as a-lactalbumin, Zn-α-2-glycoprotein, apoliprotein D, and prolactin-inducible protein.

Due to the high dynamic range between high abundant proteins and low level proteins in DL, most of the low levels in DL proteins could be identified only on the basis of one peptide using a 3D-trap MS. With the implementation of newer linear ion-trap (2D-trap) MS, much higher sensitivity was achieved and therefore significantly more proteins could be identified with more than 2 peptides. All the proteins that have been identified by 3D-trap MS were also identified by 2D-trap MS, with better quality MS spectra and better amino acid sequence coverage. Moreover, some unique proteins that had not been found by 3D-trap were identified by 2D-trap MS (Table II). (The proteins in bold face in both Table I and Table II have previously been identified as putative biomarkers for breast cancer by non-proteomic and immunochemical methods; see Reference Section.) The proteins identified by 2D-trap not only expand the families of proteins found, but also cover a wider range of functions. In addition to secreted proteins, which could be expected to play a role in the microenvironment of the tumor, these proteins can be classified as belonging to a variety of other families, which information informs the development of panels of biomarkers reflecting various aspects of the disease. As shown in Table IV, these proteins belong to various families, including enzymes/inhibitors, receptors/membrane proteins, signaling molecules, etc. Some of them have not yet been reported in plasma or biofluids and thus can have potential as novel markers of disease protection and be candidates for immunochemical assays such as ELISA.

With the increased sensitivity of the 2D-trap MS, protein expression patterns in individual subjects were able to be studied, and significant differences in protein levels among individuals were demonstrated. Preliminary 2D-trap data from individual samples also showed some promising low abundant proteins that have not been picked up before in multiple samples. Those proteins were compiled as a comprehensive list from multiple samples analyzed (Table III). Due to their low abundance, many of them were identified by one peptide in one or two samples.

### EXAMPLE II

### Biological Relevance to Breast Cancer

There are a number of proteins listed in Table I and II as mammary gland specific, or highly expressed or secreted by breast epithelial cells. Other than the secreted proteins as mentioned above, they also included polymorphic epithelial mucin, bile-salt-activated lipase, and lactadherin (BA46 antigen). The presence of mammary gland-specific proteins in DL should be correlated with the physiology and pathophysiology in the breast.

Apolipoprotein D and prolactin-inducible proteins were originally identified in large amounts in cyst fluid from women with gross cystic disease of the breast, a condition associated with increased risk of breast cancer. Both proteins and other secreted proteins in NAF and DL have potential as diagnostic markers for breast cancer and for breast cancer progression because they have been related to protein expression levels relative to hormone responsiveness and other pathophysiological conditions.

Several proteins found in DL that have also been reported in NAF have been implicated in breast cancer (in bold face in Table I). Low apolipoprotein D levels in breast tumors were associated with reduced survival. The clusterin protein was undetectable in normal breast epithelial cells, but detectable in 50% of atypical hyperplasias, intraductal and invasive carcinomas. Secreted Zn-α-2-glycoprotein was increased in serum in individuals with several cancer types, including those with breast cancer. Serum levels of a-lactalbumin were elevated in 64% of patients with breast cancer, with mean levels 2-fold greater than the control group. Prolactin-induced protein was expressed in more than 90% of human breast cancer biopsies but not in the normal mammary gland. Levels for both antitrypsin and antichymotrypsin were elevated in plasma from breast cancer patients.

Several other unique proteins found in DL have been shown in the literature to have significant biological relevance to breast cancer (in bold face in Table I and II). Calgranulin B, a member of S100 protein family of calcium-binding proteins, was present in cystic fluid from ovarian carcinomas and in serum of the corresponding patients, but absent or not detectable in fluid from benign ovarian cysts. Moreover, gene expression profiling of the breast ductal carcinomas showed that calgranulin B was abundantly expressed in invasive tumors versus ductal carcinoma in situ (DCIS).

In primary breast cancer tissues, tissue-type plasminogen activator (tPA) level correlated with nodal status and tumor grade, as well as with the receptors of estrogen and progesterone (ER and PR, respectively). Reduced tPA-mediated plasmin production was an independent adverse prognostic factor in breast cancer.

Lactadherin (BA46) is a major glycoprotein of the human milk fat globule membrane and was found to be overexpressed in human breast carcinomas. The BA46 antigens were released from the tumor and were detected in sera of the patients with breast cancer but not in those of either healthy females or from patients harboring tumors of other histological origin.

Apolipoprotein E (ApoE) is one of the key regulatory proteins in cholesterol metabolism, and was shown to influence the pathobiology of breast carcinomas. A possible link was also found between variants of the ApoE gene and breast cancer.

The CD59 glycoprotein was found to be strongly expressed by all human breast cancer tumors examined, and expression of CD59 correlated with clinicopathological features and survival of patients with breast carcinomas.

A few well-established breast cancer markers identified by immunological techniques were also identified in DL. The lysosomal protease cathepsin D was associated with increased invasiveness and metastasis in breast cancer. Cathepsin D has also been reported to be increased in serum and tumor tissue from breast cancer patients and appears to have significant prognostic value. Polymorphic epithelial mucin (mucin 1) has been identified as a breast cancer-associated antigen in breast cancer patients. Aberrantly glycosylated forms of mucin 1 were expressed in human epithelial tumors, such as breast and ovarian cancers.

Hormonal response elicited by steroid hormones (e.g., estrogen, progesterone and androgen) plays an important role in the pathological condition of the breasts. There is evidence of dross-talk between the signaling pathways of steroids, as well as with other hormonal factors, such as prolactin and growth hormone (somatotropin). Steroid hormones (e.g., estrogen, progesterone), prolactin and growth hormone were shown to be important for mammary gland development and homeostasis. Several of the potential biomarkers we found in DL were regulated by hormonal factors, such as prolactin inducible proteins, ApoD, mucin 1, somatotropin, and cathepsin D. Their expression might have an influential role in mammary gland function and etiology of breast cancer.

### EXAMPLE III

### Biological Pathways of the DL Proteins

The proteins identified in DL (Tables I, II and III) are associated with various biological pathways and functions, as indicated in Table IV. These pathways contribute not only to the normal biological functions but also to the pathological conditions of the breasts as well as to the development and progression of breast cancer. For example, steroid hormone pathways regulate many aspects of mammary gland function, specifically the etiology of breast cancer. The large number of novel proteins that have not previously been reported as being released in either normal breast function or in disease indicate that these biomarkers will be of value in understanding of protein expression and associated pathways in breast cancer and be of diagnostic significance.

An exemplary method of using the potential biomarkers disclosed herein and practicing the methods of the invention is as follows. Referring again to Table IV, the general status of a patient's health may be assessed by first examining the relative levels of the proteins shown as being associated with lipid transport and metabolism. Then, those proteins shown as being associated with the immune response and complement pathways (e.g., to look for defects in coagulation, which would indicate a clotting disorder) can be assayed. Additionally, it is valuable to examine the relative abundance of proteins associated with protein degredation, which would indicate tissue remodeling, and of the indicated steroid hormones (which, as indicated above, often stimulate breast cancer).

Others of the indicated biological pathways in Table IV are specifically associated with a cancerous state, and it is important to assess the abundance (and relative abundance) of the proteins associated therewith. For example, an increase in the levels of the proteins associated with cell adhesion and signaling and/or of cell motility and regulation could indicate a transition to metastisis. Similarly, an increase in the levels of the proteins associated with DNA transcription, regulation and repair and in those associated with oncogenic and apoptotic pathways could indicate uncontrolled growth of cancer cells.

Preferably, a panel of biomarkers would be assessed. Such a panel would include, for example, a few proteins from Table IV associated with general health, e.g., the immune response, complement pathways and inflammation, along with a few proteins indicated as being more closely associated with the cancerous state. The absolute levels of the individual proteins in the patient being tested relative to controls as well as the relative levels of the biomarkers in the panel would be informative. A control sample can be, for example, from a known non-cancerous duct of the same breast of the patient, from the non-tested breast of the patient or from the breast of a healthy individual.

Samples from individual ducts of a patient may be pooled, for example, when the status of the patient is being monitored, e.g., following a therapeutic regimen. Alternatively, it would be valuable to assay lavage samples from individual ducts so as to localize the cancerous cells, permitting a minimally invasive surgical procedure for removing the cancer.

### EXAMPLE IV

### Protein Profiling in NAF

For comparative purposes, a few NAF samples from individual patients were analyzed by shotgun proteomic approaches as described for DL. There were significant similarities for the major proteins identified between DL and NAF (Table I vs. Table V). The abundant proteins in NAF were also comparable to the published NAF proteins from the pooled patients. However, there were also several abundant proteins in the NAF samples that were not detectable in DL samples.

Based on these results from both DL and NAF and the published studies of NAF, both NAF and DL can be sources of potential breast cancer biomarkers and sites and/or diagnosis and status monitoring in individual patients. However, DL has the advantage over NAF in that the microenvironment within individual ducts can be accessed.

TABLE I
Relative abundant proteins idenified by 3D-trap MS in DL

| Protein | Accession # |
|---|---|
| Lactotransferrin | 6175096 |
| Apolipoprotein D | 114034 |
| Serum Albumin | 113576 |
| Zn-_{α}2-glycoprotein | 141596 |
| Prolactin-inducible protein | 134170 |
| Polymeric Ig receptor | 1730570 |
| Clusterin | 116533 |
| _{A}-lactalbumin | 126001 |
| Complement C4 | 116602 |
| Serotransferrin | 136191 |
| Secretogranin I | 134461 |
| Ryanodine receptor 2 | 17380312 |
| _{α}-1-antitrypsin | 1703025 |
| _{α}-1-antichymotrypsin | 112874 |
| _{α}-1-acid glycoprotein 2 | 231458 |
| _{α}-1-acid glycoprotein 1 | 112877 |
| Complement C3 | 116594 |
| beta-2-microglobulin | 114773 |
| Calgranulin B | 115444 |

TABLE II
Additional proteins identified by 2D-trap MS in DL

| Protein | Accession # |
|---|---|
| Tissue-type plasminogen activator | 137119 |
| Apolipoprotein E | 114039 |
| Clara cell phospholipid-binding protein | 112672 |
| CD59 Glycoprotein | 116021 |
| Proactivator polypeptide | 134218 |
| Lactadherin | 2506380 |
| Somatotropin (growth hormone) | 134703 |
| Cathepsin D | 115717 |
| Polymorphic epithelial mucin (mucin 1) | 547937 |
| Monocyte differentiation antigen CD14 | 115956 |
| Lysozyme C | 126615 |
| gamma-glutamyltranspeptidase 1 | 121148 |
| Bile-salt-activated lipase | 231629 |

TABLE III
Lower probability proteins identified in DL by LC-MS

| Protein | Accession # |
|---|---|
| Peripheral plasma membrane protein CASK | 6166125 |
| Myosin heavy chain | 13432177 |
| CH-TOG protein | 3121951 |
| Cytochrome C oxidase polypeptide IV | 117086 |
| Casein kinase II, alpha chain | 125266 |
| Huntingtin (Huntington's disease protein) | 1170192 |
| C—C chemokine receptor type 4 | 1705894 |
| Hemoglobin alpha chain | 122412 |
| Homeobox protein NKX-2.5 | 1708211 |
| Sterol regulatory element binding protein-2 | 3024646 |
| Active breakpoint cluster region-related protein | 5915668 |
| Guanine nucleotide exchange factor DBS | 6014924 |
| Amyloid beta A4 | 6226838 |
| Apoliprotein A-II | 114000 |
| Haptoglobin-2 | 123508 |
| A-2-macroglobulin | 112911 |
| Transthyretin (prealbumin) | 136464 |
| Calgranulin A (MRP-8) | 115442 |
| Extracellular superoxide dismutase (Cu—Zn) | 134635 |
| Prominin-like protein 1 (antigen AC133) | 13124442 |
| Ubiquitin | 12643294 |
| Zinc finger protein 8 | 141686 |
| Chromatin assembly factor 1 subunit A | 17373489 |
| Caldesmon (CDM) | 2498204 |
| DnaJ homolog subfamily A member 2 | 14916548 |
| Low affinity nerve growth factor receptor | 128156 |
| Hypothetical protein KIAA1383 | 14286071 |
| Matrix Metalloproteinase 15 (MMP-15) | 1705988 |
| Plectin 1 (PLTN) | 14195007 |
| Beta-casein | 115661 |
| alpha-s1-casein | 1345671 |
| Apoliprotein A-I | 113992 |
| Apoliprotein B-100 | 114014 |
| GA binding protein alpha (GABP-_{α}) | 729553 |
| Myb-related protein A | 1171089 |
| Transcriptional regulator ATRX | 17380440 |
| DNA-repair protein XRCC1 | 139820 |
| von Willebrand Factor | 401413 |
| Kallikrein 4 | 9296995 |
| Actin, alpha skeletal muscle | 113287 |
| Semaphorin 3B (Semaphorin V) | 8134673 |

TABLE IV
Biological pathways associated with DL proteins

| Pathway | Proteins |
|---|---|
| Lipid transport and metabolism | Apolipoprotein A-I, A-II, B-100 |
| | Apolipoprotein E |
| | Clara cell phospholipid-binding protein |
| | Aplipoprotein D |
| | bile-salt-activated lipase |
| | Zn-a2-glycoprotein |
| Immune response | a-1-acid glycoprotein 1 and 2 |
| | Moncyte differentiation antigen CD14 |
| | b-2-microglobulin |
| | Lysozyme C |
| Complement pathways | Complement C3 and C4 |
| | CD59 glycoprotein |
| Inflammation response | Calgranulin A and B |
| Protein degradation | Cathepsin D |
| | Ubiquitin |
| Cell adhesion and signaling | Lactadherin (BA46) |
| | Peripheral plasma membrane protein CASK |
| | Polymorphic epithelial mucin (mucin 1) |
| Homeostasis | a-1-antitrypsin |
| | a-1-antichymotrypsin |
| | von Willebrand factor |
| | Tissue-type plasminogen activator |
| Antioxidant defense system | Extracellular superoxide dismutase (Cu—Zn) |
| | gamma-glutamyltranspeptidase 1 |
| Cell motility and regulation | Myosin heavy chain |
| | Huntingtin |
| | Ryanodine receptor 2 |
| | Caldesmon |
| | Plectin 1 |
| Lysosomal degradation | Cathepsin D |
| | Proactivator polypeptide |
| Transport of proteins, irons | Serum albumin |
| and carbohydrate | A-lactalbumin |
| | Lactotransferrin |
| | Serotransferrin |
| | Haptoglobin-2 |
| | Transthyretin |
| | Casein, beta and alpha-s1 |
| | Polymeric Ig receptor |
| DNA transcription, regulation and repair | GA binding protein alpha |
| | Chromatin assembly factor 1 subunit A |
| | Zinc finger protein 8 |
| | transcriptional regulator ATRX |
| | DNA-repair protein XRCC1 |
| | Myb-related protein A |
| Oncogenic and apoptotic pathway | Low affinity nerve growth factor receptor |
| | Casein kinase II |
| | Clusterin |
| Steroid hormone | Kallikrein 4 |
| | Polymorphic epithelial mucin (mucin 1) |
| | Cathepsin D |
| | Secretogranin I |
| | Prolactin inducible protein |
| | Aplipoprotein D |
| | Casein kinase II |
| | Sterol regulatory element binding protein-2 |

TABLE V
Abundant proteins identified in NAF by 3D-trap MS

| Protein | Accession # |
|---|---|
| Polymeric Ig receptor | 1730570 |
| Serum Albumin | 113576 |
| Apolipoprotein D | 114034 |
| Lactotransferrin | 6175096 |
| Prolactin-inducible protein | 134170 |
| Zn-_{α}2-glycoprotein | 141596 |
| _{A}-lactalbumin | 126001 |
| _{B}-casein | 115661 |
| Clusterin | 116533 |
| Secretogranin I | 134461 |
| Complement C4 | 116602 |

### REFERENCES

1. NAF-LC/MS: Varnum, Susan M. et al., Proteomic characterization of nipple aspirate fluid: identification of potential biomarkers of breast cancer. Breast Cancer Research and Treatment 80: 87-97, 2003.
2. NAF-2D-gel: Kuerer, Henry M. et al., Identification of distinct protein expression patterns in bilateral matched pair breast ductal fluid specimens from women with unilateral invasive breast carcinoma. Cancer 95: 2276-82, 2002.
3. ApoD: Diez-Itza, Irene et al., Expression and prognostic significance of Apolipoprotein D in breast cancer. Am J Pathology 144: 310-320, 1994.
4. Prolactin-inducible protein: Clark, J W et al., The potential role for prolactin-inducible protein (PIP) as a marker of human breast cancer micrometastasis. British J Cancer 81: 1002-1008, 1999.
5. Clusterin: Redondo, Maximino et al., Overexpression of clusterin in human breast carcinoma. Am J. Pathology 157: 393-399, 2000.
6. Zn-a2-glycoprotein: Bundred, N J et al., Zinc alpha-2 glycoprotein levels in serum and breast fluids: a potential marker of apocrine activity. Eur J Cancer 27: 549-552, 1991.
7. A-Lactalbumin: Thean E T and Toh B H, Serum human alpha-lactalbumin as a marker for breast cancer. Br J Cancer 61: 773-775, 1990.
8. a1-antitrypsin and a1-antichymotrypsin: Wojtukiewicz, M Z et al., Profiles of plasma serpins in patients with advanced malignant melanoma, gastric cancer and breast cancer. Haemostasis 28: 7-13, 1998.
9. Calgranulin B: Ott, H W et al., Calgranulins in cystic fluid and serum from patients with ovarian carcinomas. Cancer Res. 63: 7507-14, 2003.
10. Calgranulin B: Seth, A et al., Gene expression profiling of ductal carcinomas in situ and invasive breast tumors. Anticancer Res. 23: 2043-51, 2003.
11. tPA: Luqmani, Y A et al., Clinical implications of urokinase and tissue type plasminogen activators and their inhibitor (PAI-1) in breast cancer tissue. Oncol Rep. 9: 645-51, 2002.
12. tPA: Chappuis, Pierre O. et al., Functional evaluation of plasmin formation in primary breast cancer. J Clinical Oncology 19: 2731-8, 2001.
13. Lactadherin: Taylor, M R et al., Lactadherin (formerly BA46), a membrane-associated glycoprotein expressed in human milk and breast carcinomas, promotes Arg-Gly-Asp (RGD)-dependent cell adhesion. DNA Cell Biol. 16: 861-9, 1997.
14. Lactadherin: Larocca, D et al., A Mr 46,000 human milk fat globule protein that is highly expressed in human breast tumors contains factor VIII-like domains. Cancer Res. 51: 4994-8, 1991.
15. ApoE: Niemi, M et al., Apolipoprotein E phenotype, cholesterol and breast and prostate cancer. J Epidemiol Community Health. 54: 938-9, 2000.
16. ApoE: Moysich, K B et al., Apolipoprotein E genetic polymorphism, serum lipoproteins, and breast cancer risk. Mol Carcinog. 27: 2-9, 2000.
17. ApoE: Zunarelli, E et al., Apolipoprotein E polymorphism and breast carcinoma: correlation with cell proliferation indices and clinical outcome. Breast Cancer Res Treat. 63: 193-8, 2000.
18. CD59: Rushmere, N K et al., Analysis of the level of mRNA expression of the membrane regulators of complement, CD59, CD55 and CD46, in breast cancer. Int J Cancer. 108: 930-6, 2004.
19. CD59: Madjd, Z et al., Loss of CD59 expression in breast tumours correlates with poor survival. J Pathol. 200: 633-9, 2003.
20. Cathepsin D: Ioachim, E et al., Clinicopathological study of the expression of hsp27, pS2, cathepsin D and metallothionein in primary invasive breast cancer. Breast. 12: 111-9, 2003.
21. Cathepsin D: Rochefort, H et al., Cathepsin D in breast cancer: mechanisms and clinical applications, a 1999 overview. Clin Chim Acta. 291: 157-70, 2000.
22. Mucin 1: Diaz, L K et al., Expression of epithelial mucins Muc1, Muc2, and Muc3 in ductal carcinoma in situ of the breast. Breast J. 7: 40-5, 2001.
23. Mucin 1: Seregni, E et al., Hormonal regulation of MUC1 expression. Int. J Biol Markers. 14: 29-35, 1999.
24. Mucin 1: Mitchell, S et al., Androgen-dependent regulation of human MUC1 mucin expression. Neoplasia. 4: 9-18, 2002.
25. Somatotropin: Laban, C et al., The GH-IEF-I axis and breast cancer. Trends Endocrinol Metab. 14: 28-34, 2003.
26. Somatotropin: van Garderen, E et al., Morphogenic and tumorigenic potentials of the mammary growth hormone/growth hormone receptor system. Mol Cell Endocrinol. 197: 153-65, 2002.
27. Somatotropin: Wennbo, H and Tornell, J, The role of prolactin and growth hormone in breast cancer. Oncogene. 19: 1072-6, 2000.
28. Hormonal regulation: Harding, C et al., Hormonally-regulated proteins in breast secretions are markers of target organ sensitivity. Br J Cancer 82: 354-360, 2000.

While the present invention has been described in conjunction with a preferred embodiment, one of ordinary skill, after reading the foregoing specification, will be able to effect various changes, substitutions of equivalents, and other alterations to the compositions and methods set forth herein. It is therefore intended that the protection granted by Letters Patent hereon be limited only by the definitions contained in the appended claims and equivalents thereof.

What is claimed is:

1. A method for assessing the presence of a cancerous or precancerous lesion in a breast of a patient, said method comprising the steps of:
a) obtaining a ductal fluid sample from at least one duct of a breast of a patient;
b) determining the level of abundance of a plurality of markers in said sample, wherein at least one of the markers is selected from the group consisting of markers listed in Tables I, II and III;
c) determining the level of abundance of said plurality of markers in a control sample; and
d) comparing the level of abundance of said plurality of markers in the patient sample to the level of abundance of said plurality of markers in the control sample,
wherein a significant difference in the level of abundance of said plurality of markers in said patient sample compared to the normal level is an indication of the presence of a cancerous or precancerous lesion in a breast of said patient.

2. A method for assessing the presence of a cancerous or precancerous lesion in a breast of a patient, said method comprising the steps of:
a) obtaining a sample of a bodily fluid from a patient;
b) determining the relative level of abundance of a plurality of markers in said sample, wherein at least two of the markers are selected from the group consisting of markers listed in Tables I, II and III;
c) determining the relative level of abundance of said plurality of markers in a control sample; and
d) comparing the relative level of abundance of said plurality of markers in the patient sample to the relative level of abundance of said plurality of markers in the control sample,
wherein a significant difference in the relative level of abundance of said plurality of markers in said patient sample compared to the control sample is an indication of the presence of a cancerous or precancerous lesion in a breast of said patient.

3. The method of claim 2, wherein said sample is a ductal fluid sample from at least one duct of a breast of said patient.

4. The method of claim 2, wherein said sample is nipple aspirate fluid from a breast of said patient.

5. The method of claim 2, wherein said sample is a blood sample.

6. The method of claim 2, wherein, in steps b and c, the relative level of abundance of said plurality of markers is determined using an assay selected from the group consisting of an antibody based assay, a protein array assay and a mass spectrometry based assay.

7. The method of claim 1 or claim 2, wherein said control sample is non-cancerous breast cells from said patient.

8. The method of claim 1 or claim 2, wherein said control sample is non-cancerous breast cells from a healthy subject.

9. The method of claim 1 or claim 2, wherein said control sample levels of abundance of said plurality of markers are determined from a standard table or curve.

10. The method of claim 1 or claim 2, wherein the level of abundance of said plurality of markers is determined by detecting the amount of marker protein present in the sample.

11. The method of claim 1 or claim 2, wherein the level of abundance of said plurality of markers is determined by detecting the amount of mRNA that encodes a marker protein present in the sample.

12. The method of claim 1 or claim 2, wherein said ductal fluid sample is free of ductal fluid from any other duct of the breast.

13. The method of claim 1 or claim 2, said method further comprising the step of correlating a positive indication of the presence of a cancerous or precancerous lesion in a breast of said patient with an individual duct of the breast.

14. The method of claim 1 or claim 2, wherein said plurality of markers is greater than three.

15. The method of claim 1 or claim 2, wherein said plurality of markers is greater than five.

16. A method of selecting a composition for inhibiting breast cancer in a patient, the method comprising the steps of:
a) obtaining a sample comprising cancer cells from the patient;
b) separately exposing aliquots of the sample to a plurality of test compositions;
c) comparing the relative level of abundance of a plurality of markers in each aliquot of said sample, wherein at least two of the markers are selected from the group consisting of markers listed in Tables I, II and III; and
d) selecting at least one of the test compositions that modifies the relative level of abundance of the plurality of markers in the aliquot exposed to that test composition, compared to the other test compositions.

17. A kit for diagnosing the presence of a cancerous or precancerous lesion in a breast of a patient, the kit comprising reagents for carrying out the method of claim 1 or claim 2.

18. A kit for diagnosing the presence of a cancerous or precancerous lesion in a breast of a patient, the kit comprising a plurality of antibodies, wherein at least two of the antibodies specifically bind with proteins corresponding to at least two markers selected from the group consisting of markers listed in Tables I, II and III.

19. A kit for assessing the suitability of one or more test compounds for inhibiting breast cancer in a patient, the kit comprising: a) one or more test compounds; and b) a reagent for assessing the relative level of abundance of a plurality of markers, wherein at least two of the markers are selected from the group consisting of markers listed in Tables I, II and III.

20. A method of identifying a potential breast cancer biomarker, said method comprising the steps of:
a) obtaining a plurality of ductal fluid samples from a plurality of donors, each sample being from at least one duct of a breast of a donor and each said sample being only from an individual said donor, wherein said plurality of donors comprise healthy individuals and high risk patients and/or cancer patients;
b) determining a profile of protein abundance in each of said samples;
c) comparing said protein abundance profiles of said high risk patients and/or cancer patients with said protein abundance profiles of said healthy individuals; and
d) identifying any proteins that are present in the abundance profiles of said high risk patients and cancer patients but not in, or at a reduced level in, the abundance profiles of said healthy individuals,
wherein any protein so identified is a potential breast cancer biomarker.
